(19) United States
(12) Patent Application Publication
(10) Pub. No.: US 2021/0030490 A1
(43) Pub. Date: Feb. 4, 2021

(54) WIRE-DISPLACEMENT DETECTION DEVICE AND MEDICAL MANIPULATOR

(71) Applicant: OLYMPUS CORPORATION, Tokyo (JP)

(72) Inventor: Shinpei MIYAHARA, Tokyo (JP)

(73) Assignee: OLYMPUS CORPORATION, Tokyo (JP)

(21) Appl. No.: 17/076,082

(22) Filed: Oct. 21, 2020

Related U.S. Application Data
(63) Continuation of application No. PCT/JP2018/016762, filed on Apr. 25, 2018.

(51) Int. Cl. A61B 34/20; G01D 5/12; A61B 34/30; A61B 34/00

(52) U.S. Cl. CPC A61B 34/20; G01D 5/12; A61B 2562/0223; A61B 34/71; A61B 34/30

(57) ABSTRACT

A wire-displacement detection device includes: a wire that drives a movable part through longitudinal movement thereof; a linear scale member that is shaped like a cylinder for allowing the wire to pass therethrough in a longitudinal direction thereof, the linear scale member including a first region not fixed to the wire and a second region fixed to the wire; a scale part disposed in the first region of the linear scale member; and a sensor that is disposed at a position facing the scale part, the sensor detecting longitudinal movement of the scale part.

### CROSS-REFERENCE TO RELATED APPLICATIONS

This is a continuation of International Application PCT/JP2018/016762, with an international filing date of Apr. 25, 2018, which is hereby incorporated by reference herein in its entirety.

### TECHNICAL FIELD

The present invention relates to a wire-displacement detection device and a medical manipulator.

### BACKGROUND ART

There is a well-known technique used in manipulator drive devices for driving a joint provided at the distal end of a flexible tube by means of a tractional force of a motive-power transmitting wire that is disposed in the tube along the longitudinal direction thereof. With this technique, a displacement sensing wire that is linked to the motive-power transmitting wire near the joint is guided to the proximal end of the tube through a sensing-wire guide part disposed parallel to the motive-power transmitting wire, and the displacement of the sensing wire is detected by a magnetic sensor at the proximal end of the tube, whereby it is possible to detect the displacement of the joint (refer to, for example, PTL 1).

There is another well-known technique in which two reflecting parts having different reflectivities are arranged in a predetermined pattern on the outer surface of a cylindrical member along the longitudinal direction thereof, said cylindrical member being fixed to a wire, so that the displacement of the wire is detected on the basis of the difference between the amounts of light reflected at the respective reflecting parts (refer to, for example, PTL 2).

### CITATION LIST

### Patent Literature

### {PTL 1}

Publication of Japanese Patent No. 5325621

### {PTL 2}

Publication of Japanese Patent No. 5284837

### SUMMARY OF INVENTION

One aspect of the present invention is directed to a wire-displacement detection device comprising: a wire that drives a movable part through longitudinal movement thereof; a linear scale member that is shaped like a cylinder for allowing the wire to pass therethrough in a longitudinal direction thereof, the linear scale member comprising a first region not fixed to the wire and a second region fixed to the wire; a scale part disposed in the first region of the linear scale member; and a sensor that is disposed at a position facing the scale part, the sensor detecting longitudinal movement of the scale part.

Another aspect of the present invention is directed to a medical manipulator comprising: an insertion section inserted into a body; a movable part provided at a distal end of the insertion section; a wire that drives the movable part through longitudinal movement thereof; a drive device for driving the wire; a linear scale member that is shaped like a cylinder for allowing the wire to pass therethrough in a longitudinal direction thereof, the linear scale member comprising a first region not fixed to the wire and a second region fixed to the wire; a scale part disposed in the first region of the linear scale member; and a sensor that is disposed at a position facing the scale part, the sensor detecting longitudinal movement of the scale part.

### BRIEF DESCRIPTION OF DRAWINGS

FIG. 1 is a side elevational view showing one example of a medical manipulator according to one embodiment of the present invention.

FIG. 2 is a longitudinal sectional view partially showing an insertion section of the medical manipulator in FIG. 1.

FIG. 3 is a transverse section, taken along A-A of the insertion section in FIG. 2.

FIG. 4 is a transverse section, taken along B-B of the insertion section in FIG. 2.

FIG. 5 is a perspective view partially showing one example of a detection unit of a drive device included in the medical manipulator in FIG. 1.

FIG. 6 is a longitudinal sectional view showing the detection unit in FIG. 5.

FIG. 7 is a longitudinal sectional view showing a modification of the detection unit in FIG. 5.

FIG. 8 is a perspective view showing a modification of the detection unit in FIG. 5.

### DESCRIPTION OF EMBODIMENTS

A drive device 4 and a medical manipulator 1 according to one embodiment of the present invention will now be described with reference to the drawings.

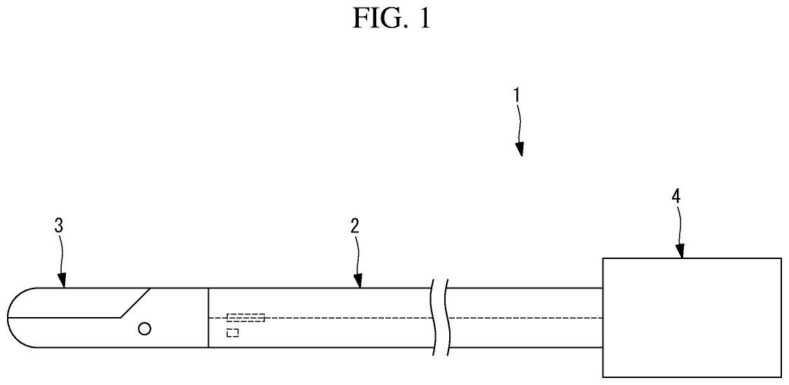

As shown in FIG. 1, the medical manipulator 1 according to this embodiment includes: an elongated flexible insertion section 2; an end effector (movable part; although FIG. 1 shows forceps as the end effector, the end effector is not limited to forceps and may be a bending mechanism or the like) 3 provided at a distal end of that insertion section 2; and the drive device 4 according to this embodiment for driving the end effector 3.

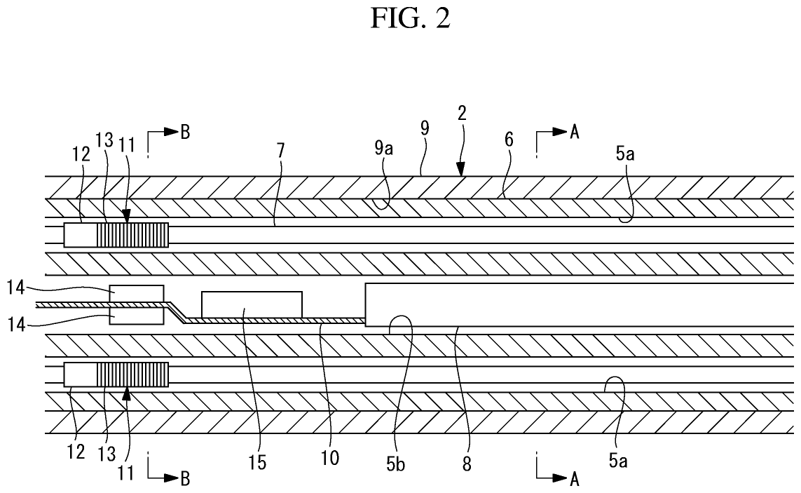

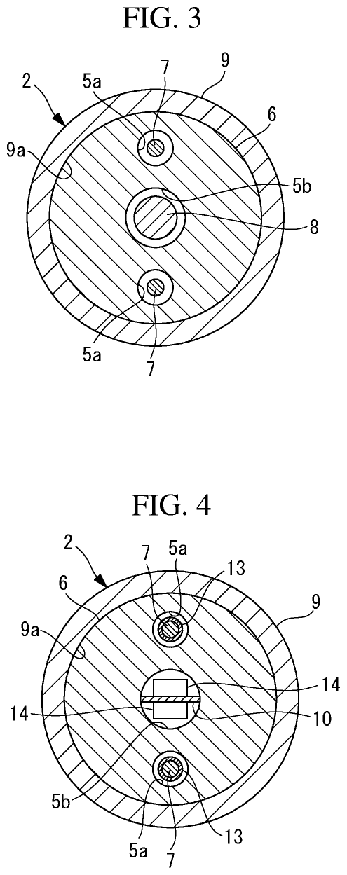

As shown in FIGS. 2 to 4, the insertion section 2 includes: a multi-lumen tube 6 including a plurality of channels 5a and 5b; a cable 8 inserted into the channel 5b at the center; and a cylindrical blade tube 9 including a channel 9a into which the multi-lumen tube 6 is inserted.

The drive device 4 includes: an operating unit (not shown in the figure) that is provided at a proximal end of the insertion section 2 and that is operated by an operator; a wire 7 that is disposed in each of the channels 5a of the multi-lumen tube 6, that links the operating unit and the end effector 3, and that transmits a force applied in the operating unit to the end effector 3; and a detection unit 11 for detecting the displacement of the wire 7.

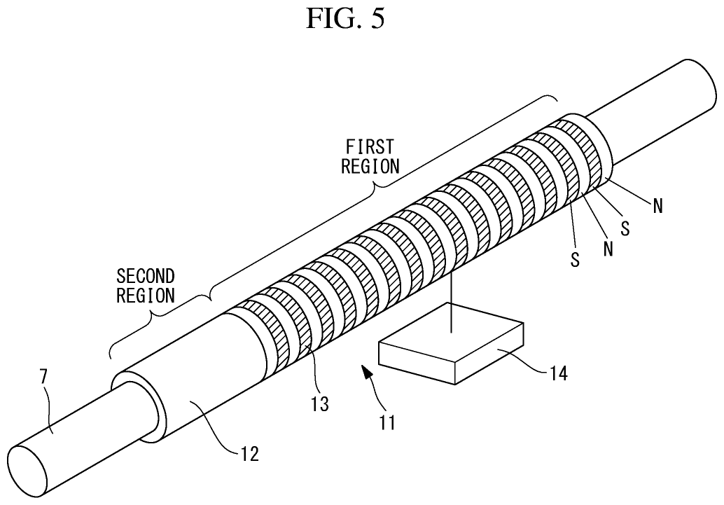

As shown in FIG. 5, the detection unit 11 includes: a linear scale member 12 fixed at an intermediate position in the longitudinal direction of any of the wires 7; and a sensor 14 disposed at a position facing a scale part 13 of the linear scale member 12. As shown in, for example, FIG. 5, the scale part 13 is formed of magnetized N poles and S poles that are alternately arranged at predetermined intervals. The sensor 14 is a magnetic sensor for detecting magnetism.

In this embodiment, the linear scale member 12 is formed in a cylindrical shape so as to allow a wire 7 to pass therethrough in the longitudinal direction. The linear scale member 12 includes, at one end thereof in the longitudinal direction, a first region in which the scale part 13 is provided and, at the other end thereof in the longitudinal direction, a second region in which the scale part 13 is not provided.

The first region has a longer longitudinal dimension than the second region.

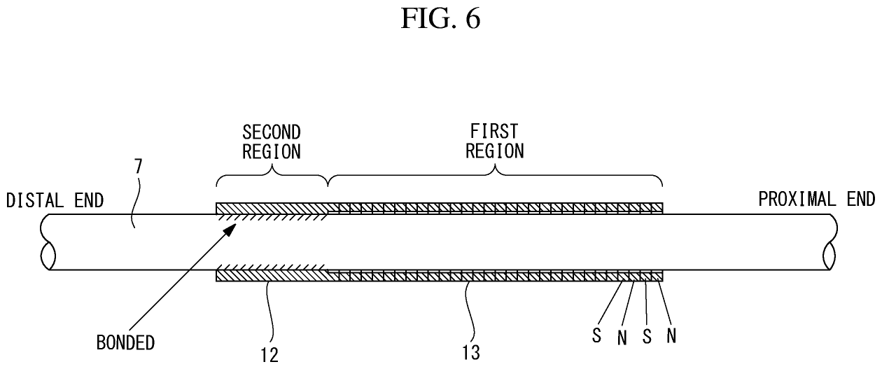

As shown in FIG. 6, the linear scale member 12 is not bonded to the wire 7, which passes through the interior thereof, in the first region and is bonded to the wire 7 in the second region. The other end of the wire 7, which includes the second region, is disposed at a position that is closer to the end effector 3 than the one end, which includes the first region, is.

The sensor 14 is mounted on, for example, a flexible board 10, and the flexible board 10 is connected to the cable 8, which is introduced via the channel 5b at the center of the multi-lumen tube 6. By doing so, power is supplied to the sensor 14 via the cable 8, and a signal detected by the sensor 14 is output to the proximal end of the medical manipulator 1 via the cable 8. Reference sign 15 in FIG. 2 denotes an A/D converter (ADC) mounted on the flexible board 10.

The operation of the drive device 4 and the medical manipulator 1 according to this embodiment with the above-described structure will be described below.

In order to carry out treatment of body tissue by using the medical manipulator 1 according to this embodiment, the insertion section 2 is inserted into a channel, such as an endoscope or an overtube, is advanced until it protrudes from an end effector channel, and is disposed at a position facing the affected area.

The end effector 3 is driven by pulling the wires 7 by operating the operating unit of the drive device 4 in this state, thus allowing the operator to treat the body tissue.

When a wire 7 moves in the longitudinal direction as a result of being pulled, the linear scale member 12 fixed to the wire 7 also moves in the longitudinal direction, whereby it is possible to detect magnetism of the scale part 13, provided in the linear scale member 12, by means the sensor 14 serving as a magnetic sensor.

Here, the scale part 13 is formed of magnetized N poles and S poles that are alternately arranged at predetermined intervals. Therefore, when the scale part 13 moves in the longitudinal direction of the wire 7 relatively to the sensor 14, the magnitude of magnetism detected by the sensor 14 varies, whereby it is possible to detect the displacement of the wire 7.

Also, in this case, according to the drive device 4 and the medical manipulator 1 of this embodiment, the linear scale member 12 is partitioned into the first region and the second region, the second region is bonded to the wire 7, and the first region is supported so as to be movable relative to the wire 7 without being bonded to the wire 7. Therefore, even when the wire 7 is stretched by a tractional force, the first region can be prevented from being stretched. Also, this embodiment is advantageous in that because the scale part 13 is provided in the first region, which is not affected by stretch of the wire 7, the displacement of the wire 7 can be detected with high accuracy.

In other words, attaching the linear scale member 12 directly to the wire 7 for driving the end effector 3 eliminates the necessity for additionally providing a displacement sensing wire and a guide for the sensing wire, making it possible to enhance the accuracy with which the displacement of the wire 7 is detected while still ensuring a small diameter of the insertion section 2.

In addition, because the first region is made to have a larger lengthwise dimension than the second region, in which the linear scale member 12 is bonded to the wire 7, it is possible to ensure a large scale part 13.

In addition, because the second region, in which the linear scale member 12 is bonded to the wire 7, is disposed closer to the distal end than the first region is, the length of the wire 7 from the end effector 3 to the second region can be kept small, whereby it is possible to suppress a decrease in the detection accuracy of the displacement due to stretch of the wire 7 at that portion.

Note that although, in the drive device 4 and the medical manipulator 1 according to this embodiment, the second region, in which the linear scale member 12 is bonded to the wire 7, is set in a region including the other end, which is at the opposite side from the one end at which the first region is provided, the location of the second region is not limited to said position, and the second region may be partially provided at an intermediate position in the longitudinal direction of the linear scale member 12.

In addition, although the scale part 13 is provided only in the first region, the location of the scale part 13 is not limited to said position, and a portion of the scale part 13 may be provided in the second region. This provides an advantage in that by ensuring a long scale part 13, it is possible to widen the area within which the displacement can be detected.

In this case, it is preferable that the range of a value detected by the sensor 14 be wider when the value is detected by the scale part 13 in the first region than when in the second region. By doing so, a detected value with a high S/N ratio can be obtained. This provides another advantage in that even when the second region is erroneously detected, the operator can determine by seeing the detected value range that the second region is detected unintentionally, whereby it is possible to prevent erroneous detection. Note that the magnitude relationship may be arbitrary, provided that the ranges of the values detected by the sensor 14 in the first and second regions do not overlap each other.

The sensor 14 is realized by a magnetic sensor, and N poles and S poles are alternately arranged in the scale part 13 in this embodiment. Instead of this, the sensor 14 may be realized by a photo sensor, and the scale part 13 may be realized by alternately arranging regions having different reflectivities.

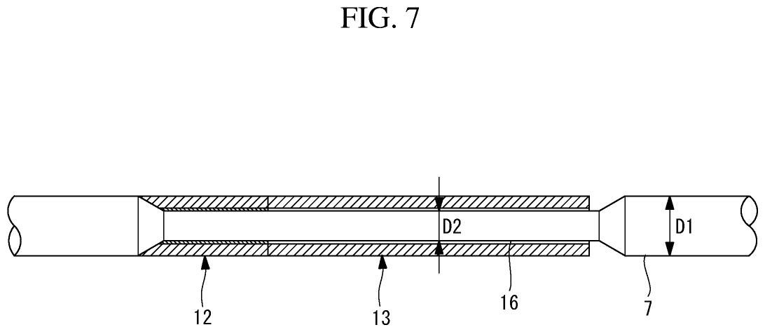

In addition, as shown in FIG. 7, each of the wires 7 in this embodiment may include, at a longitudinal portion thereof, a small-diameter section 16 that has an outer diameter D2 smaller than an outer diameter D1 at the other longitudinal portion of the wire 7 and that has a longitudinal dimension equal to or larger than that of the linear scale member 12, so that the linear scale member 12 may be disposed within the longitudinal dimension of this small-diameter section 16. The outer diameter of the linear scale member 12 in this case can be made equal to or smaller than the outer diameter D1 at the other portion, whereby it is possible to reduce the diameter of the insertion section 2.

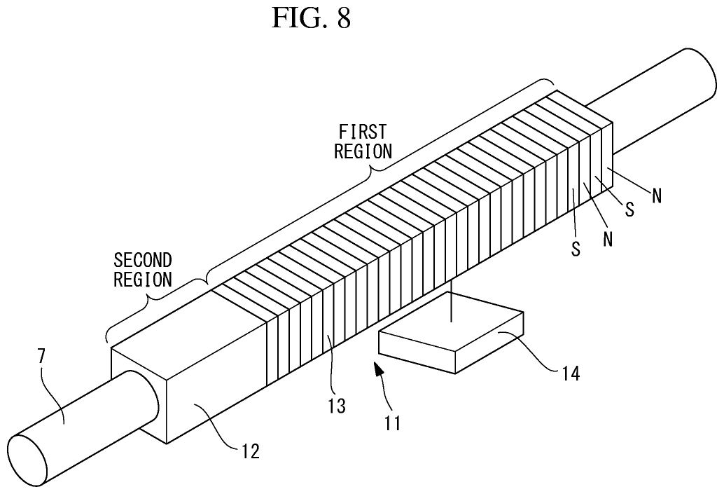

In addition, although the linear scale member 12 is cylindrical in shape so as to allow the wire 7 to pass therethrough in this embodiment, the shape of the linear scale member 12 is not limited to said shape. As shown in, for example, FIG. 8, a linear scale member 12 that has a flat surface facing the sensor 14 may be employed.

In other words, the linear scale member 12 may be formed so as to have a rectangular transverse section, as shown in FIG. 8. Note that the transverse section of the linear scale member 12 is not limited to a rectangular cylindrical shape but may be an external shape obtained by partially cutting a circular shape with a straight line, or the linear scale member 12 may have a polygonal external shape with a hole through which the wire 7 is made to pass.

In addition, in this case, it is preferable that a rotation stopper be provided so that the flat surface of the linear scale member 12 is located parallel to the sensor 14.

This provides an advantage in that even when the position of the wire 7 varies in a direction orthogonal to the direction in which the wire 7 is opposed to the sensor 14, it is possible to prevent fluctuations of the distance between the sensor 14 and the flat surface of the linear scale member 12, whereby stable detection is achieved.

As a result, the above-described embodiment leads to the following aspects.

One aspect of the present invention is directed to a drive device including: a wire that drives a movable part through longitudinal movement thereof; a sensor that detects the longitudinal movement of the wire; and a linear scale member that has an elongated shape so as to be disposed along the longitudinal direction of the wire and that includes a scale part the movement of which is detected by the sensor, wherein the linear scale member includes a first region that includes one end of the linear scale member and that is not fixed to the wire and a second region that is at least partially fixed to the wire, and the scale part is disposed in at least the first region.

According to this aspect, when the wire moves in the longitudinal direction as a result of a tractional force being applied to the wire, the movable part is driven due to the longitudinal movement of the wire. In this case, the movement of the wire is detected by the sensor by using the scale part that is included in the linear scale member provided on the wire. However, because the first region in which the scale part is disposed is not fixed to the wire and is thus movable relative to the wire, the scale part is not affected even when the wire is stretched.

More specifically, because the linear scale member with which the movement of the wire is detected is attached to the wire itself for driving the movable part, it is not necessary to separately prepare a displacement sensing wire, whereby it is possible to achieve a small diameter of the insertion section. Furthermore, because the scale part of the linear scale member attached to the wire for driving the movable part is not affected by stretch of the wire, the movement of the wire, i.e., the movement of the movable part can be detected with high accuracy.

In the above-described aspect, the length of the second region in the longitudinal direction may be smaller than or equal to one half of the total length of the linear scale member in the longitudinal direction.

By doing so, the length of the second region is reduced, whereby it is possible to reduce the area affected by stretch of the wire.

In the above-described aspect, the second region may be disposed closer to the other end than the center position of the linear scale member in the longitudinal direction is.

By doing so, it is possible to ensure that the first region is a long region, whereby it is possible to increase the length of the scale part not affected by stretch of the wire.

In the above-described aspect, the second region may include the other end.

By doing so, it is possible to ensure that the first region is a long region, whereby it is possible to increase the length of the scale part not affected by stretch of the wire.

In the above-described aspect, the wire may include, at a portion in the longitudinal direction, a small-diameter section that has a smaller outer diameter at the portion than at another portion and that has a longitudinal dimension equal to or larger than that of the linear scale member, and the linear scale member may be disposed within the longitudinal dimension of the small-diameter section.

By doing so, it is possible to reduce the dimension of the linear scale member disposed in the small-diameter section, said dimension being in a direction orthogonal to the longitudinal direction, whereby the diameter of the insertion section can be further reduced.

In the above-described aspect, the scale part may be provided in the first region and the second region, and the detected value range in the case where the sensor sets the first region as a detection subject may be out of the detected value range in the case where the sensor sets the second region as a detection subject.

By doing so, even in the case where the sensor detects a region deviated from the scale part, such erroneous detection can be prevented because a detected value that is output in that case differs from a detected value in the case where the sensor detects the scale part.

In the above-described aspect, a surface of the linear scale member, the surface facing the sensor, may be a flat surface.

By doing so, even when the sensor is shifted in a direction orthogonal to the longitudinal axis of the linear scale member, it is possible to suppress fluctuations in detected values.

Another aspect of the present invention is directed to a medical manipulator including: an insertion section inserted into a body; a movable part provided at a distal end of the insertion section; and one of the above-described drive devices.

In the above-described aspect, the second region may be disposed closer to the movable part than the first region is.

By doing so, the linear scale member can be fixed to the wire at a position closer to the movable part. This leads to a shorter wire length from the movable part to the second region, thereby making it possible to reduce the frequency of occurrence of a detection error due to stretch of that portion.

The present invention affords an advantage in that the displacement of a movable part at a distal end of an insertion section can be detected with high accuracy while still reducing the diameter of the insertion section.

### REFERENCE SIGNS LIST

1 Medical manipulator
2 Insertion section
3 End effector (movable part)
4 Drive device
7 Wire
12 Linear scale member
13 Scale part
14 Sensor
16 Small-diameter section

1. A wire-displacement detection device comprising:
a wire that drives a movable part through longitudinal movement thereof;
a linear scale member that is shaped like a cylinder for allowing the wire to pass therethrough in a longitudinal direction thereof, the linear scale member comprising a first region not fixed to the wire and a second region fixed to the wire;
a scale part disposed in the first region of the linear scale member; and
a sensor that is disposed at a position facing the scale part, the sensor detecting longitudinal movement of the scale part.

2. The wire-displacement detection device according to claim 1, wherein a length of the second region in the longitudinal direction is smaller than or equal to one half of a total length of the linear scale member in the longitudinal direction.

3. The wire-displacement detection device according to claim 2, wherein the second region is disposed closer to the movable part than a center position of the linear scale member in the longitudinal direction is.

4. The wire-displacement detection device according to claim 3, wherein the second region includes one end of the linear scale member, the one end being on the movable part side.

5. The wire-displacement detection device according to claim 1,
wherein the wire includes, at a portion in the longitudinal direction, a small-diameter section that has a smaller outer diameter at the portion than at another portion and that has a longitudinal dimension equal to or larger than that of the linear scale member, and
the linear scale member is disposed within the longitudinal dimension of the small-diameter section.

6. The wire-displacement detection device according to claim 1,
wherein the scale part is provided in the first region and the second region, and
a detected value range in a case where the sensor sets the first region as a detection subject is out of a detected value range in a case where the sensor sets the second region as a detection subject.

7. The wire-displacement detection device according to claim 1, wherein a surface of the scale part, the surface facing the sensor, is a flat surface.

8. The wire-displacement detection device according to claim 1, wherein the second region is disposed closer to the movable part than the first region is.

9. The wire-displacement detection device according to claim 1, wherein, in the scale part, N poles and S poles are alternately arranged at predetermined intervals.

10. The wire-displacement detection device according to claim 1, wherein, in the scale part, regions having different reflectivities are alternately arranged.

11. A medical manipulator comprising:
an insertion section inserted into a body;
a movable part provided at a distal end of the insertion section;
a wire that drives the movable part through longitudinal movement thereof;
a drive device for driving the wire;
a linear scale member that is shaped like a cylinder for allowing the wire to pass therethrough in a longitudinal direction thereof, the linear scale member comprising a first region not fixed to the wire and a second region fixed to the wire;
a scale part disposed in the first region of the linear scale member; and
a sensor that is disposed at a position facing the scale part, the sensor detecting longitudinal movement of the scale part.

12. The medical manipulator according to claim 11, wherein a length of the second region in the longitudinal direction is smaller than or equal to one half of a total length of the linear scale member in the longitudinal direction.

13. The medical manipulator according to claim 12, wherein the second region is disposed closer to the movable part than a center position of the linear scale member in the longitudinal direction is.

14. The medical manipulator according to claim 13, wherein the second region includes one end of the linear scale member, the one end being on the movable part side.

15. The medical manipulator according to claim 11,
wherein the wire includes, at a portion in the longitudinal direction, a small-diameter section that has a smaller outer diameter at the portion than at another portion and that has a longitudinal dimension equal to or larger than that of the linear scale member, and
the linear scale member is disposed within the longitudinal dimension of the small-diameter section.

16. The medical manipulator according to claim 11,
wherein the scale part is provided in the first region and the second region, and
a detected value range in a case where the sensor sets the first region as a detection subject is out of a detected value range in a case where the sensor sets the second region as a detection subject.

17. The medical manipulator according to claim 11, wherein a surface of the scale part, the surface facing the sensor, is a flat surface.

18. The medical manipulator according to claim 11, wherein the second region is disposed closer to the movable part than the first region is.

19. The medical manipulator according to claim 11, wherein, in the scale part, N poles and S poles are alternately arranged at predetermined intervals.

20. The medical manipulator according to claim 11, wherein, in the scale part, regions having different reflectivities are alternately arranged.
